(19) United States
(12) Patent Application Publication
(10) Pub. No.: US 2007/0298015 A1
(43) Pub. Date: Dec. 27, 2007

(54) Treatment of Muscular Dystrophy with Mobilized Peripheral Blood Pluripotent Cells

(75) Inventors: Marc D. Beer, Sudbury, MA (US); Morey Kraus, Harvard, MA (US)

Correspondence Address: CLARK & ELBING LLP, 101 FEDERAL STREET, BOSTON, MA, 02110, US

(73) Assignee: VIACELL, INC., Cambridge, MA (US)

(21) Appl. No.: 11/587,764

(22) PCT Filed: Apr. 28, 2005
(86) PCT No.: PCT/US05/14750

§ 371 Date: Apr. 4, 2007

Related U.S. Application Data
(60) Provisional application No. 60/565,953, filed on Apr. 28, 2004.

(51) Int. Cl. A61K 35/12; A61P 21/00

(52) U.S. Cl. 424/93.7

(57) ABSTRACT

The invention features methods for treating a patient suffering from muscular dystrophy by administering mobilized pluripotent cells isolated from peripheral blood.

### BACKGROUND OF THE INVENTION

Muscular dystrophy represents a family of inherited diseases of the muscles. Some forms affect children (e.g., Duchenne dystrophy) and are lethal within two to three decades. Other forms present in adult life and are more slowly progressive. The genes for several dystrophies have been identified, including Duchenne dystrophy (caused by mutations in the dystrophin gene) and the teenage and adult onset Miyoshi dystrophy or its variant, limb girdle dystrophy 2B or LGMD-2B (caused by mutations in the dysferlin gene). These are “loss of function” mutations that prevent expression of the relevant protein in muscle and thereby cause muscle dysfunction. Mouse models for these mutations exist, either arising spontaneously in nature or generated by inactivation or deletion of the relevant genes. These models are useful for testing therapies that might replace the missing protein in muscle and restore normal muscle function.

Differentiated muscle is composed of multi-nucleated cells or myofibers that have an extraordinary capacity to regenerate. This regenerative capacity exists because muscle possesses primitive muscle precursor cells (muscle pluripotent cells and somewhat more mature cells known as “satellite cells”). These cells lie dormant in muscle and can be activated to make new mononucleated muscle cells (myoblasts) that can adhere to one another and fuse to make new, multi-nucleated myotubes, as well as the more mature muscle cells (that are again multinucleated). Because myofibers arise from the fusion of individual myoblasts, a protein made by one muscle cell is readily accessible to be shared with neighboring muscle cells lacking that protein if the two cells fuse into the same myotube.

Inherent in this concept of myoblast fusion and muscle regeneration is the possibility of cell therapy of muscle diseases. The fusion of a myoblast capable of making a muscular dystrophy protein with muscle cells that lack the protein should correct the deficiency in the resulting myotube. That is, the normal nucleus in the normal myoblasts replaces a gene missing in the dystrophic muscle cells thus achieving gene and protein replacement through cell therapy.

Partridge and colleagues demonstrated more than a decade ago that a mixed population of muscle precursor cells capable of making normal dystrophin protein could fuse into muscle of the mdx mouse that lacks dystrophin and thereby partially replace the missing protein (Partridge et al., Nature 337:176-179, 1989). In the seminal experiments of Partridge, it was not clear precisely what populations of the muscle precursor cells had the capacity to achieve this effect. At least six human trials of myoblast therapy were undertaken in Duchenne and Becker dystrophy patients, using direct intramuscular injections of myoblasts; that none were effective might be interpreted to mean that myoblasts were not sufficiently undifferentiated to participate effectively in muscle cell therapy. This observation stimulated the search for muscle pluripotent cells.

Over the last two years, several muscle biologists have had encouraging initial success in isolating putative muscle pluripotent cells. Moreover, these studies have documented not only that the primitive muscle precursors can fuse into injured muscle to make new muscle, but also that such pluripotent cells or pluripotent-like cells have an extraordinary capacity to circulate in the blood and then to leave the blood to enter sites of focal muscle injury in response to unidentified myotropic factors. Recently it has become apparent that cells with features of muscle pluripotent cells may be present in tissues thought previously to be primarily hematogenic, such as the bone marrow. It was recently reported that a population of primitive cells identified by the presence of a multi-drug resistance transporter as a “side population” (SP) fraction of cells in either the bone marrow or muscle itself could be delivered to dystrophic mdx muscle following tail vein injection (Gussoni et al., Nature 401:390-394, 1999). That these cells included primitive pluripotent cells was strongly suggested by the finding that the same injection could populate muscle tissue with enough normal muscle cells to restore dystrophin expression in up to 10% of myofibers and, at the same, repopulate the bone marrow of previously irradiated recipient mice. Analogous findings were subsequently reported by Lee et al., J. Cell Biol. 150:1085-1099, 2000 and Torrente et al., J. Cell Biol. 152:335-348, 2001.

One criterion for defining a pluripotent cell as a muscle pluripotent cell is the capacity to differentiate to form myoblasts and thereby augment some aspect of muscle regeneration or repair. Typically, the expression of a previously missing protein (such as, for example, dystrophin) after muscle pluripotent cell infusion provides prima facie evidence that a muscle pluripotent cell is present. In addition, it is likely that a candidate set of pluripotent cells will express the surface antigen CD34 and perhaps other primitive cell surface markers such as AC133, but not lineage markers, such as c-kit or the hematopoietic marker CD45.

Mobilized peripheral blood progenitor cells have become an increasingly used alternative to bone marrow as a source for pluripotent cells used in allogeneic transplantation procedures (see Bensinger and Storb, Rev. Clin. Exp. Hematol. 5:67-86, 2001 and Ringden et al., Blood 94:455-64, 1999. Studies have demonstrated that engraftment of neutrophils, red blood cells and platelets is faster with peripheral blood cells compared to marrow.

### SUMMARY OF THE INVENTION

In a first aspect, the present invention features a method for treating a patient suffering from muscular dystrophy that includes administering to the patient mobilized pluripotent cells isolated from peripheral blood. In one embodiment, the mobilized pluripotent cells are mononuclear. In another embodiment, the peripheral blood is obtained from and allogeneic donor. In another embodiment, the pluripotent cells are injected into the blood stream of the patient. In another embodiment, the pluripotent cells are injected into the muscle of the patient. In yet another embodiment, prior to administration the pluripotent cells are treated to increase their number.

In another aspect, the invention features a cell transplant method for treating a patient suffering from muscular dystrophy, while reducing rejection of the transplanted cells, that includes the steps of: providing a preparation of mobilized pluripotent cells isolated from the peripheral blood of an allogeneic donor; treating the patient with an immunosuppressive agent; reconstituting the immune system of the patient using cells of the preparation; and administering cells of the preparation to treat the patient's muscular dystrophy. In one embodiment, the method further includes expanding cells of the preparation in vitro and repeatedly administering the expanded cells of the preparation to the patient to treat the patient's muscular dystrophy.

In another aspect, the invention features a cell transplant method for treating a patient suffering from muscular dystrophy that includes the steps of: providing a preparation of mobilized pluripotent cells isolated from the peripheral blood, preferably from an allogeneic donor; purifying the preparation to provide another preparation of mobilized pluripotent cells; and administering the preparation of purified cells to the patient to treat the patient's muscular dystrophy. In one embodiment, the method further includes expanding cells of the preparation in vitro and repeatedly administering the expanded cells of the preparation to the patient to treat the patient's muscular dystrophy. In another embodiment, the purified preparation is enriched in CD34⁺ cells. In another embodiment, the purified preparation is enriched in CD34⁻/Sca-1⁺ cells. In yet another embodiment, the purified preparation is enriched in L-selectin⁺ cells, which can be either CD34⁺ or CD34⁻. Preferably, the purified preparation is enriched in cells that are CD34⁻/Sca-1⁺/L-selectin⁺.

Definitions

By “mobilized pluripotent cells” are meant pluripotent cells originating from bone marrow that are induced to migrate to peripheral blood by treating a donor subject with a chemotherapeutic agent (e.g., a cytoreductive agent such as cyclophosphamide), a cytokine (e.g., a growth factor such as granulocyte colony-stimulating factor, interleukin-3, or stem cell factor), or a combination thereof.

By “muscular dystrophy” or “MD” is meant any one of a group of muscle diseases in which there is a recognizable pattern of inheritance. They are marked by weakness and wasting of selected muscles, where affected muscle fibers degenerate and are replaced by fatty tissue. The dystrophies can be classified according to the patient's age at onset, distribution of the weakness, progression of the disease, and mode of inheritance. The most common form is Duchenne muscular dystrophy (DMD), which is inherited as a sex-linked recessive gene. Other muscular dystrophies include Becker's MD, facioscapulohumeral MD, limb-girdle MD, Emery-Dreifuss MD, myotonic dystrophy, and myotonia congenita.

By “pluripotent cell” is meant a cell having the ability to give rise to two or more cell types of an organism.

### DETAILED DESCRIPTION

Muscular dystrophy is a group of inherited disorders, all characterized by variable degrees and distribution of muscle wasting and weakness. In the present invention, a method of treating muscular dystrophy is provided that features diagnosing muscular dystrophy in a patient; obtaining a cell population of mobilized pluripotent cells that have been isolated from peripheral blood, preferably the blood of an allogeneic donor; optionally expanding the cell population of pluripotent cells by treatment with one or more agents that increase their number; optionally purifying the pluripotent cell population before and/or after cell population expansion; and administering the pluripotent cells to the patient, either by intravenous injection into the patient's bloodstream or by injection into the patient's muscle tissue. The other features and advantages of the invention will become apparent from the following detailed description, which is given for the purpose of describing preferred embodiments of the invention. The following description does not limit the scope of the invention, which will become apparent to those skilled in the art.

Diagnosing Muscular Dystrophy

The treatment methods of the present invention can be used to treat any human patient, whether a child or an adult, who suffers from one of the forms of muscular dystrophy as described herein. Any of the methods can further include a step in which muscular dystrophy is first diagnosed. Typically, a diagnosis of MD is made after a family history review and a physical examination, which may include one of more of the following: a blood test; electromyography, ultrasonography; muscle biopsy; and genetic testing.

When a biopsy is performed, the muscle sample obtained can be used for tests that identify dystrophin or other markers associated with specific forms of muscular dystrophy. For example, abnormalities in the expression or structure of proteins important for the function of muscle tissue, such as, for example, those listed in Table 1, can be identified by the use of antibodies, such as, for example, antibodies against epitopes in the N-terminal or C-terminal domain of dystrophin.

Identification of Biological Markers Used in Diagnosing Muscular Dystrophy

At least 30 different forms of muscular dystrophy have been identified to date (see Vainzof and Zatz, Braz. J. Med. Biol. Res. 36:543-555, 2000 and Table 1). Duchenne (DMD) and Becker (BMD) muscular dystrophies are allelic conditions caused by mutations in the dystrophin gene at Xp21 (Koenig et al., Cell 53:219-228, 1988; and Hoffman et al., Cell 51:919-928, 1987). The limb-girdle muscular dystrophies (LGMD) include a heterogeneous group of progressive disorders mainly affecting the pelvic and shoulder girdle musculature, ranging from severe forms with onset in the first decade of life and rapid progression, to milder forms of later onset and slower. Inheritance may be autosomal dominant (LGMD1) or recessive (LGMD2). The autosomal dominant forms are relatively rare and represent less than 10% of all LGMD. The six autosomal dominant LGMD forms are: LGMD1A at 5q22, coding for the protein myotilin, LGMD1B at 1q11, coding for lamin A/C, LGMD1C at 3p25 coding for caveolin-3, LGMD1D at 6q23, LGMD1E at 7q, and LGMD1F at 5q31.

Ten autosomal recessive forms have been mapped up to now and most of their protein products have been identified. Four of them, mapped at 17q21, 4q12, 13q12 and 5q33, respectively code for a-sarcoglycan (a-SG), β-SG, g-SG and d-SG, that are glycoproteins of the SG subcomplex of the dystrophin-glycoprotein complex (DGC). Mutations in these genes cause LGMD2C, 2D, 2E and 2F, respectively, and constitute a distinct subgroup of LGMD, i.e., the sarcoglycanopathies (see Bushby, Human Molecular Genetics 8:1875-1882, 1999; and Zatz et al., Current Opinion in Neurology 13:511-517, 2000). Among the clinically milder forms, LGMD2A, at 15q15.1, codes for calpain 3, LGMD2B, at 2p31, codes for dysferlin, and LGMD2G, at 17q11-12, codes for the sarcomeric telethonin. Recently, the fukitin-related protein (FKRP) gene, mapped at 19q13.3, was identified as the gene responsible for the LGMD2I form, as well as the severe form of congenital muscular dystrophy type 1 (CMD1C); the protein TRIM32 has been identified as the gene product of the LGMD2H form at 9q31-33. LGMD2J was recently described in the Finish population as the result of autosomal recessive mutations in the titin gene.

The dystrophin gene is located in the subregion 21 of the short arm of the X-chromosome. The size of the gene is 3.0 Mb, and it is the largest known human gene. Despite this large size, only 14 kb regions in total encode the dystrophin protein, with those encoding regions divided into 79 exons that are distributed throughout the gene (see Roberts et al., Genomics 16:536-538, 1993). The gene also contains eight distinct promoter regions (see Nishio et al., J. Clin. Invest. 94:1073-1042, 199; Ann and Kunkel, Nature Genet. 3:283-291, 1993; and D'Souza et al., Hum. Mol. Genet. 4:837-842, 1995).

TABLE 1
Proteins involved in different genetic neuromuscular disorders

| | Muscular Dystrophy | Mode of | | |
|---|---|---|---|---|
| Proteins | Phenotypes | Inheritance | Gene Location | MIM |
| Dystrophin | DMD/BMD | XL | Xp21 | 310200 |
| α-Sarcoglycan | LGMD2D | AR | 17q12 | 600119 |
| β-Sarcoglycan | LGMD2E | AR | 4q12 | 604286 |
| γ-Sarcoglycan | LGMD2C | AR | 13q12 | 253700 |
| δ-Sarcoglycan | LGMD2F | AR | 5q33-34 | 601287 |
| Dysferlin | LGMD2B/Miyoshi myopathy | AR | 2p13 | 253601 |
| Caveolin-3 | LGMD1C | AD | 3p25 | 601253 |
| α2-Laminin | Merosin-deficient CMD | AR | 6q2 | 156225 |
| Collagen VI | Ullrich CMD, Bethlem | AR | 21q22 | 254090 |
| | myopathy | | | |
| Calpain 3 | LGMD2A | AR | 15q15.1 | 253600 |
| TRIM32 | LGMD2H | AR | 9q31-34 | 254110 |
| Myotubularin | Myotubular myopathy | XL | Xq28 | 310400 |
| Fukutin | Fukuyama CMD | AR | 9q31-33 | 253800 |
| FKRP | LGMD2I/CMD1C | AR | 19q1 | 606596 |
| POMFnT1 | Muscle-eye-brain CMD | AR | 1p32-34 | 253280 |
| POMT1 | Walker-Warburg CMD | AR | 9q34 | 236670 |
| Titin | LGMD2J | AR | 2q | 159000 |
| Myotilin | LGMD1A | AD | 5q22-34 | 601954 |
| Telethonin | LGMD2G | AR | 17q11-12 | 101800 |
| Actin | Nemaline myopathy | AD/AR | 1q42 | 256030 |
| Tropomyosin 3 | Nemaline myopathy | AD/AR | 1q21-23 | 161800 |
| Tropomyosin 2 | Nemaline myopathy | AD | 9q13 | 190990 |
| Nebulin | Nemaline myopathy | AR | 2q21-22 | 256030 |
| Troponin T1 | Nemaline myopathy | AR | 19q13 | 190990 |
| Emerin | Emery-Dreifuss MD | XL | Xq28 | 310300 |
| Lamin A/C | LGMD1B | AD | 1q11-21 | 159001 |
| SMN | Spinal muscular atrophy | AR | 5q11-13 | 253300 |
| AR/AD—autosomal dominant and recessive inheritance; BMD—Becker muscular dystrophy; CMD—congenital muscular dystrophy; DMD—Duchenne muscular dystrophy; FKRP—fukutin-relted protein; LGMD—limb-girdle muscular dystrophy; SMN—survival motor neural protein; XL—X-linked inheritance; | | | | |
| # MIM number—Mendelian Inheritance in Man (see catalogs of autosomal dominant, autosomal recessive and X-linked phenotypes provided on-line as OMIMby McKusick-Nathans Institute for Genetic Medicine, Johns Hopkins University, Baltimore, MD and National Center for Biotechnology Information, National Library of Medicine, Bethesda, MD 2000, http://www.ncbi.nlm.nih.gov/omim/) | | | | |

As approximately six out of ten DMD or BMD patients have abnormalities such as large loss or multiplication in dystrophin gene, genetic diagnosis of DMD and BMD can performed by Southern blotting using cDNAs as probes for fragments of the dystrophin gene. Most genetic abnormalities found in DMD/BMD patients involve loss of genetic information, with as much as several kb being absent. Abnormalities in dystrophin gene detected by Southern blotting are concentrated on two “hot-spots” in the gene, and a multiplex polymerase chain reaction (PCR) technique has been designed for genetic diagnosis, which can conveniently identify a deletion using two PCR systems by focusing on 19 exons in these hot-spots (see Chamberlain et al., Nucleic Acids Res. 16:11141-11156, 1988; and Beggs et al., Hum. Genet. 86:45-48, 1990).

PCR techniques used for the diagnosis of muscular dystrophy can also include multiplex PCR (see Hofstra et al., Hum. Mutat. 23:57-66, 2004). The genetic analysis techniques used in U.S. Pat. Nos. 5,552,282 and 6,040,142 for the diagnosis of Myotonic muscular dystrophy and spinal muscular atrophy, respectively, can also be used.

Genetic testing can include the use of single condition amplification/internal primer (SCAIP) sequencing, to examine entire genes (e.g., the dystrophin gene) to find multiple deleterious variations, thereby providing more than just one type of diagnosis (see Flanigan et al., Am. J. Hum. Genet. 72:931-9, 2003). Any genetic testing performed as part of a method of the invention can be guided by the gene location information provided in Table 1.

Pluripotent Cell Mobilization

In accordance the first aspect of the present invention, the concentration of the pluripotent cells in the peripheral blood of a donor can be increased by administering an agent (e.g., by subcutaneous or intravenous injection) to induce mobilization of pluripotent cells to the peripheral blood of the donor (see, for example, Champlin et al., Blood 95:3702-9, 2000). A number of such agents are known and include cytokines, such as granulocyte-colony stimulating factor (G-CSF), granulocyte-macrophage colony stimulating factor (GM-CSF), interleukin (IL)-7, IL-3, IL-12, stem cell factor (SCF), and flt-3 ligand; chemokines such as IL-8, Mip-1α, and Groβ, and the chemotherapeutic agents of cylcophosamide (Cy) and paclitaxel. These agents differ in their time frame to achieve pluripotent cell mobilization, the type of pluripotent cell mobilized, and efficiency. A preferred mobilizing agent is a colony stimulating factor, such as G-CSF. A typical dosage of G-CSF in a subject is about 1 to 100 μg/Kg per day, preferably about 5 to 25 μg/Kg per day, and most preferably, about 5 to 15 μg/Kg per day, for about 5 to about 10 days.

In one example, the mobilized pluripotent cell donor (for example, a sibling, a parent, or another family members such as an aunt, an uncle or a cousin) is selected on the basis of the donor-versus-patient pluripotent cell alloreactivity. The cell donor may not need to be the only pluripotent cell donor, therefore allowing the banking of alloreactive clones with known allospecificity ahead of time of pluripotent cell administration. Donor selection can be based on typing of HLA-A, B, C, DR loci to be carried out on the recipient. The donor must be at least genotypically HLA-A, B, C, DR haploidentical to the patient, but can differ for 2-3 HLA alleles on the unshared haplotype.

At least one week before the patient starts conditioning for the transplant, the donor will receive G-CSG (either filgrastim or lenograstim) at either 12 mg/kg/day in a continuous subcutaneous infusion or 8 mg/kg/day in two push doses (every 12 hours) for 7 days. On the fourth day of this course of G-CSF, if the circulating CD34 count is >40 mL, the donor will start leukapheresis, which will continue for three consecutive days. Normal access is through a peripheral vein; central venous access may be required in exceptional cases. Each leukapheresis product is processed by a cell selection device, such as, for example, the automated CliniMACS® apparatus (Miltenyi Biotec GmbH), and cryopreserved until used. The mobilized cell population can be highly purified in another purification step, as described by Lang et al. in Blood 101:1630-6, 2003, before administration to the patient to reduce the likelihood of graft vs. host disease. Purification of the mobilized cells can be performed separate from, before, or after a cell expansion procedure. Purification of mobilized cells may also be useful to increase the likelihood of their incorporation into the muscle tissue of the patient, as was reported for cells that are CD34⁻/Sca-1⁺/L-selectin⁺ (see Torrente et al., J. of Cell Biol. 162:511-20, 2003).

Expansion of Mobilized Peripheral Blood Pluripotent Cells

In general, any mobilized precursor cell population suitable for transformation into myoblasts can be isolated and used in accordance with the present invention. The pluripotent cells so obtained can be expanded under cell growth conditions, i.e., conditions that promote proliferation (“mitotic activity”) of the cells. For example, the mobilized cells can be expanded by methods described in U.S. Pat. No. 5,674,750, U.S. Pat. No. 5,925,567, and U.S. Pat. No. 6,338,942. After expansion, the cells are then administered to the patient. If desired, the expanded pluripotent cell population can be purified before administration to the patient to enrich the expanded population in those cells characterized by markers known to be important for cell incorporation into muscle tissue.

Administration of Mobilized Pluripotent Cells by Systemic Infusion

Mobilized pluripotent cells are prepared and isolated as described above. Therapy is likely to require multiple infusions of either mobilized pluripotent cell preparations isolated by a suitable leukapheretic technique or various enriched and expanded sub-populations of the those cells. Several infusions of cells can be administered over time, e.g., one on day one, a second on day five, and a third on day ten. After the initial ten-day period, there can be a period of time without cell administration, e.g., two weeks to 6 months, after which time the ten-day administration protocol can be repeated.

Whether administered as a single infusion therapy or multiple infusion therapies, it is likely that the recipient will require immunosuppression. The protocols followed for this will follow the precedents now used in human transplantation for bone marrow replacement (i.e., cell transplantation), with such agents as cyclosporin A and FK506.

Administration of Mobilized Pluripotent Cells by Intramuscular Injections

Another possible administration route for mobilized pluripotent cells, or expanded sub-populations of these mobilized cells, is via direct injection into muscle. It is well established that under certain circumstances direct intramuscular injection of muscle precursor cells can lead to the incorporation of the donor cells into the host, with partial replacement of a missing protein (e.g., dystrophin in the mdx mice). It is also well established that direct myoblast injections into humans with muscular dystrophy is safe, although such myoblasts have not been incorporated significantly into the host muscles. Accordingly, it is believed that sub-populations of mobilized pluripotent cells that are more primitive than myoblasts will fuse into host muscle and thereby permit protein replacement and muscle regeneration. This is inherently less efficient than IV injection for widespread distribution of the injected cells, and thus would almost certainly require multiple injections into multiple muscles, with treatment interruption intervals lasting from 2 week to 6 months, or as otherwise determined by the attending physician.

### OTHER EMBODIMENTS

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

What is claimed is:

1. A method for treating a patient suffering from muscular dystrophy comprising administering to said patient mobilized pluripotent cells isolated from peripheral blood.

2. The method of claim 1, wherein said peripheral blood is obtained from an allogeneic donor.

3. The method of claim 1, wherein said pluripotent cells are injected into the bloodstream of said patient.

4. The method of claim 1, wherein said pluripotent cells are injected into the muscle of said patient.

5. The method of claim 1, wherein prior to administration, said pluripotent cells are treated to increase their number.

6. A cell transplant method for treating a patient suffering from muscular dystrophy comprising the steps of:
a) providing a first preparation of mobilized pluripotent cells isolated from the peripheral blood of an allogeneic donor;
b) purifying said cells to provide a second preparation; and
c) administering cells of said second preparation to treat said patient's muscular dystrophy.

7. The method of claim 6, further comprising expanding cells of said second preparation in vitro and repeatedly administering said expanded cells to said patient to treat said patient's muscular dystrophy.

8. The method of claim 6, wherein said second preparation is enriched in CD34⁺ cells.

9. The method of claim 6, wherein said second preparation is enriched in CD34⁻/Sca-1⁺ cells.

10. The method of claim 6, wherein said second preparation is enriched in L-selectin⁺ cells.
